(19) United States
(12) Patent Application Publication
(10) Pub. No.: US 2019/0160244 A1
(43) Pub. Date: May 30, 2019

(54) ADVANCED FLOW BRONCHOSCOPE

(71) Applicants: Trent PARRY, Salt Lake City, UT (US); Kyle THORNLEY, South Jordan, UT (US); Joshua BURTON, West Valley City, UT (US); John NELSON, St. George, UT (US); Albert PARK, Salt Lake City, UT (US)

(72) Inventors: Trent PARRY, Salt Lake City, UT (US); Kyle THORNLEY, South Jordan, UT (US); Joshua BURTON, West Valley City, UT (US); John NELSON, St. George, UT (US); Albert PARK, Salt Lake City, UT (US)

(21) Appl. No.: 16/090,519

(22) PCT Filed: Apr. 3, 2017
(86) PCT No.: PCT/US2017/025769

§ 371 Date: Oct. 1, 2018

Related U.S. Application Data
(60) Provisional application No. 62/316,988, filed on Apr. 1, 2016.

(51) Int. Cl. A61M 16/04; A61B 1/267; A61B 17/02; A61B 17/34

(52) U.S. Cl. CPC A61M 16/0463; A61B 1/2676; A61B 17/0218; A61B 2017/00809; A61B 17/3462; A61B 2017/345; A61B 17/3423

(57) ABSTRACT

A bronchoscope includes a main shaft extending from a proximal end to a distal end having an aperture. The proximal end of the bronchoscope includes a plurality of ports. One port is a ventilation port in fluid communication with the main shaft via a ventilation tube. The ventilation tube includes a first portion and a second portion, wherein the second portion extends from the first portion to the main shaft. The second portion is disposed at angle of approximately 60-70 degrees relative to the first portion. Another port may be configured to receive an adapter that includes a first member and a second member, wherein the second member is rotatable relative to the first member to relocate a tool received by the adapter within the main shaft.

### CROSS-REFERENCE TO RELATED APPLICATIONS

This application is a non-provisional of and claims priority to U.S. Provisional Application No. 62/316,988, filed on Apr. 3, 2016, the contents of which are incorporated herein by reference.

### BACKGROUND OF THE INVENTION

There are currently two methods of performing endoscopic procedures within the respiratory tract. One method uses a flexible scope and the other a rigid scope. The present invention relates to rigid scopes. Rigid scopes generally include a solid metal tube that branches at the proximal end into several ports. The main shaft provides access, airflow, and a place for anesthetic delivery.

The majority of commercially available rigid bronchoscopes have a single union at the proximal end where the branches and main shaft merge (FIG. 1). Anything introduced to the system (e.g. light, oxygen, tools) must pass this single location. While several different bronchoscope designs exist, they all follow this same pattern. Physicians complain that the current design negatively affects ventilation capabilities. Inefficient ventilation often results in pausing the procedure and removing the device until oxygen levels return to normal. Such disturbances can increase the time and cost of the procedure as well as put the patient at more risk. There are often abrasions of the respiratory tract caused by the insertion and removal of the bronchoscope. The more the physician has to remove and reinsert the device, the greater the risk to the patient. The present invention introduces a new design of the distal end of the bronchoscope that optimizes oxygen flow, reducing the need to pause during procedures. Without the need to remove and reinsert the bronchoscope as often, patient risk decreases and overall care improves.

### SUMMARY OF THE INVENTION

The present invention improves upon rigid bronchoscopes used for visualization of the respiratory tract. More specifically, the present invention allows for increased oxygen delivery to patients undergoing treatment, which in turn reduces the need for pauses during procedures. Due to the decrease in patient risk and overall improved care, the present invention will replace currently used rigid bronchoscopes.

The present invention relates to a novel design of the bronchoscope that improves oxygen delivery and ventilation. More specifically, the invention includes a hollow stainless steel tube with a series of branches on the proximal end, wherein the various branches merge with the main shaft in different locations allowing for increased oxygen flow. Additionally, airflow is improved by a rotating adapter that repositions inserted tools so as to not block airflow through the main insertion port. This allows the user to maintain a closed system while clearing the main shaft for improved airflow.

The various ports located at the proximal end provide inputs for various tools associated with bronchoscopic procedures. The ventilation port, according to one embodiment of the present invention, is designed to deliver air in a direct route to the main shaft. This is achieved using an angled wall within the ventilation port that guides the air into a committed ventilation shaft. The committed ventilation shaft then connects directly to the main shaft just distal to the intersection of the different branches. There is included, also, a port for tools to be inserted, a port for insertion of a light, and a telescopic port. The main shaft ends in a feature that holds the adapter in place in a manner that seals the environment. The adapter is configured to intersect with existing telescopes and telescopic tools used in bronchoscopy.

According to some embodiments of the present invention, the adapter may be used to seal the system and provide a rotating interface to move a telescope to the edge of the main shaft, improving direct airflow. The adapter comprises two parts joined with a screw. The top piece contains a single hole, centered, while the bottom has a curved slot that moves the inserted device (telescope etc.) to the edge of the design. The adapter is designed to fit into the main insertion port of the present invention as well as other current bronchoscope models.

In one embodiment, the invention provides a bronchoscope including a main shaft defined by a hollow tube extending from a proximal end of the bronchoscope to an open distal end of the bronchoscope. The bronchoscope also includes a first port defined at the proximal end that is in fluid communication with the main shaft via a first tube. The first port is configured to receive one or more operative tools. The bronchoscope also includes a second port defined at the proximal end that is in fluid communication with the main shaft. The second port defines a ventilation port to deliver air to a patient via the main shaft. The bronchoscope also includes a ventilation tube fluidly coupling the second port to the main shaft. The ventilation shaft is fluidly separated from the first tube by a dividing wall.

In another aspect, the invention provides an adapter for a tool port of a bronchoscope that is continuous with a main shaft of the bronchoscope, including a first member configured to be coupled to the tool port of the bronchoscope, and a second member rotationally coupled to the first member. The second member includes a port to receive a tool, and rotation of the second member relative to the first member moves the port from a first position to a second position.

In another aspect, the invention provides a bronchoscope including a main shaft defined by a hollow tube extending from a proximal end of the bronchoscope to an open distal end of the bronchoscope. The bronchoscope also includes a first port defined at the proximal end that is in fluid communication with the main shaft via first tube. The first port is configured to receive one or more operative tools. An adapter that is coupled to the first port includes a first member configured to be coupled to the first port of the bronchoscope, and a second member rotationally coupled to the first member. The second member includes a port to receive a tool, and rotation of the second member relative to the first member moves the tool port from a first position to a second position. The bronchoscope also includes a second port defined at the proximal end that is in fluid communication with the main shaft. The second port defines a ventilation port to deliver air to a patient via the main shaft. The bronchoscope also includes a ventilation tube fluidly coupling the second port to the main shaft. The ventilation shaft is fluidly separated from the first tube by a dividing wall.

Other aspects of the invention will become apparent by consideration of the detailed description and accompanying drawings.

### BRIEF DESCRIPTION OF THE DRAWINGS

FIG. 1 is a perspective view of a prior art bronchoscope.

FIG. 2 is a perspective view of a bronchoscope according to an embodiment of the present invention.

FIG. 3 is a side view of a proximal end of the bronchoscope illustrated in FIG. 2.

FIG. 4 is a side cross-sectional view of the proximal end of the bronchoscope illustrated in FIG. 2.

FIG. 5 is a first perspective view of a cross-section of the proximal end of the bronchoscope.

FIG. 6 is a second perspective view of a cross-section of the proximal end of the bronchoscope.

FIG. 7 is a second cross-sectional view of the proximal end of the bronchoscope.

FIG. 8A is an illustration of an airflow analysis within the prior art bronchoscope of FIG. 1.

FIG. 8B is an illustration of an airflow analysis within the bronchoscope of FIG. 2.

FIG. 9 is a perspective view of a proximal end of a bronchoscope with an adapter according to another embodiment of the invention.

FIG. 10 is a perspective view of an adapter of the bronchoscope illustrated in FIG. 9.

FIG. 11 is an exploded view of the adapter.

FIG. 12 is a perspective view of the adapter with a top portion shown in phantom lines.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

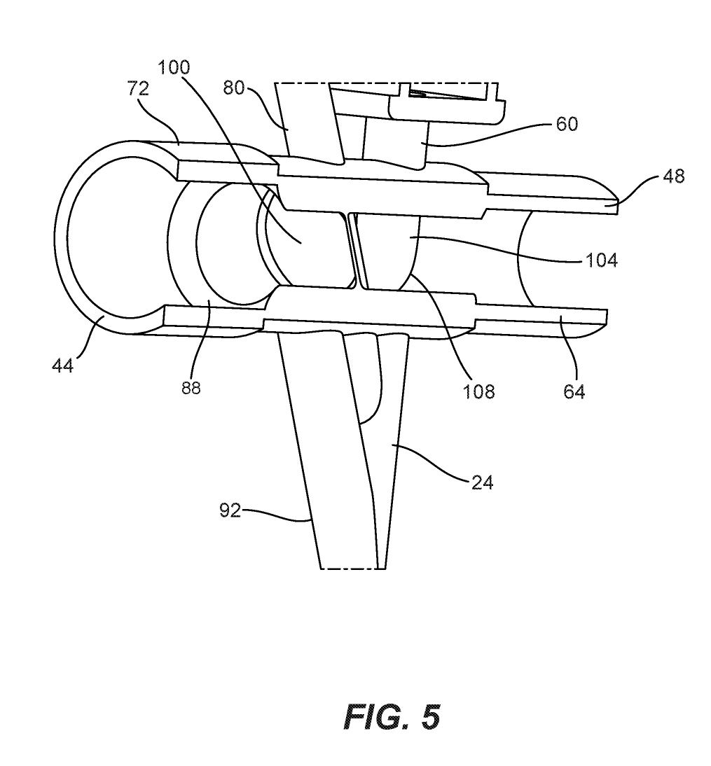

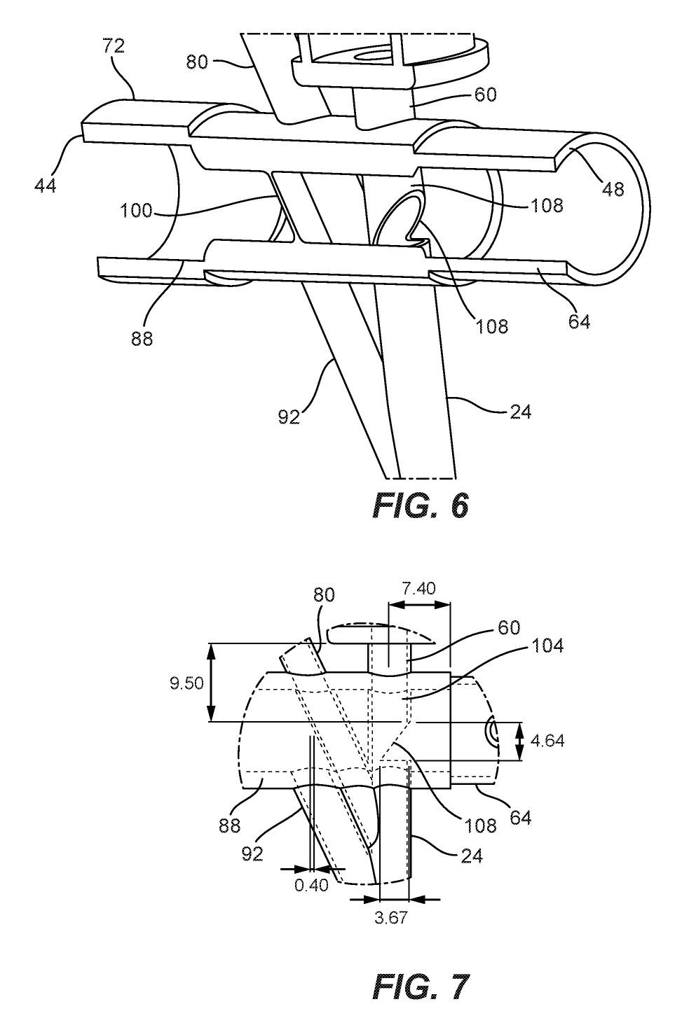

FIGS. 2-7 illustrate a bronchoscope 20 that optimizes oxygen delivery to a patient. The bronchoscope 20 includes a main shaft 24 defined by a hollow tube extending from a proximal end 28 to an open distal end 32 having a primary outlet aperture 36. The proximal end 28 includes a plurality of ports 40. In the illustrated embodiment, the proximal end 28 includes a plurality of access ports, for example, a ventilation port 44, a light or auxiliary port 48, a tool access port 52, and a main insertion port 56.

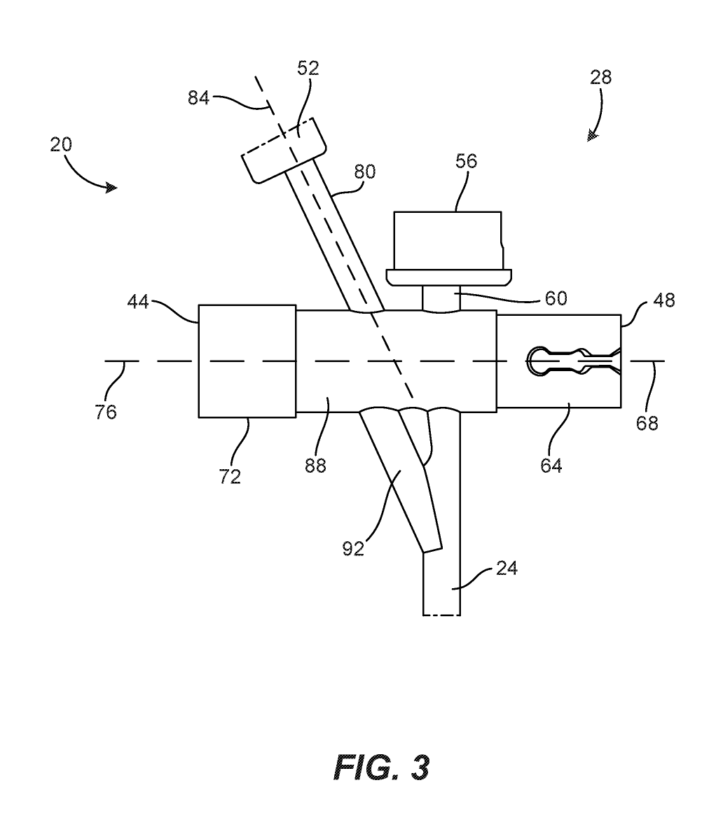

With reference to FIG. 3, the main insertion port 56 is engageable with accessory devices (e.g., a telescope, a video stylet, etc.) to removably lock the accessory device to and seal the accessory device within the main insertion port 56. A main insertion tube 60 fluidly couples the main insertion port 56 to the main shaft 24, and extends towards and is continuous with the main shaft 24.

The auxiliary port 48 receives illumination devices or other tools. An auxiliary port tube 64 fluidly couples the auxiliary port 48 to the main shaft 24, and extends along an auxiliary port axis 68 that is disposed at an oblique angle relative to the main shaft 24. In the illustrated embodiment, the auxiliary port axis 68 is disposed at an angle of approximately 90 degrees relative to the main shaft 24. However, in other embodiments, the angle may be varied.

The ventilation port 44 communicates air (e.g., oxygen) into the main shaft 24. A ventilation port tube 72 fluidly couples the ventilation port 44 to the main shaft 24, and extends along a ventilation port axis 76 disposed at an oblique angle relative to the main shaft 24. In the illustrated embodiment, the ventilation port axis 76 is disposed at an angle of approximately 90 degrees relative to the main shaft 24 opposite to the auxiliary port axis 68. That is, the ventilation port axis 76 and the auxiliary port axis 68 are parallel and collinear, with the tubes 64, 72 extending in opposed directions from the main shaft 24. However, in other embodiments, the orientation of the ventilation port axis 68 may be varied.

The tool access port 52 receives tools (e.g., puncture needles, injection cannulae, suction tubes, cotton applicators, sponge holders, etc.) to be used during operation of the bronchoscope. A tool access port tube 80 fluidly couples the tool access port 52 to the main shaft 24, and extends along a tool access port axis 84 that is disposed at an oblique angle relative to the main shaft 24. In the illustrated embodiment, the tool access port axis 84 is disposed in a plane defined by the ventilation port axis 76 and the main shaft 24 at an angle of approximately 20-70 degrees relative to the ventilation port axis 76.

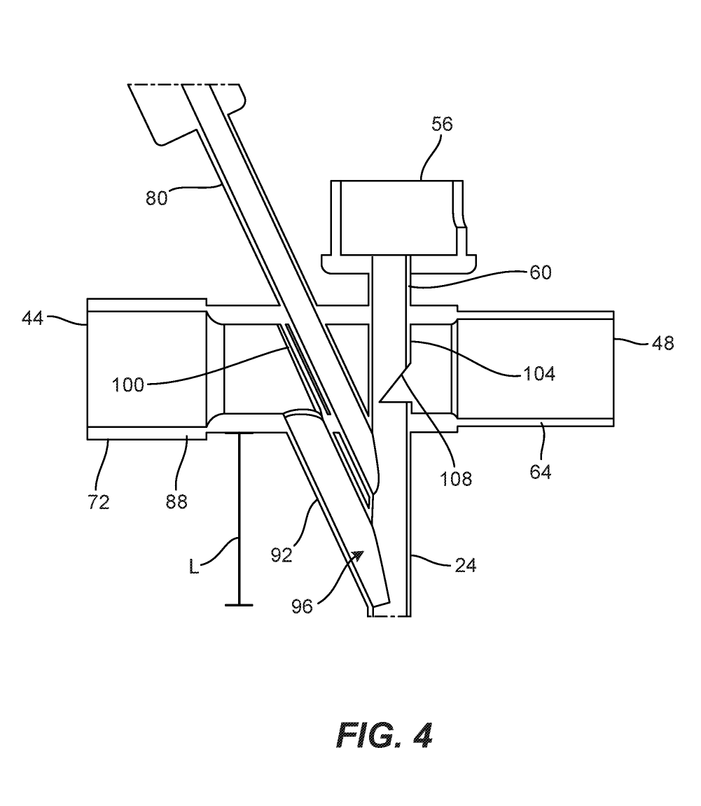

With reference to FIGS. 4-6, the ventilation port tube 72 includes a first portion 88 defined by a cylindrical sidewall such that the first portion 88 extends along the ventilation port axis 68. The ventilation port tube 72 also includes a second portion or ventilation shaft 92 fluidly coupling the first portion 88 to the main shaft 24 at an airflow port 96 on the main shaft 24. The ventilation shaft 92 is disposed at an airflow angle of approximately 55-75 degrees relative to the first portion 88 (i.e., 15-35 degrees relative to the main shaft 24). In another embodiment, the airflow angle is approximately 60-70 degrees relative to the first portion 88 (i.e., 20-30 degrees relative to the main shaft 24). In yet another embodiment, the airflow angle is approximately 63-65 degrees relative to the first portion 88 (i.e., or 25-27 degrees relative to the main shaft 24). The airflow port 96 is disposed an insertion length L of approximately 10-20 mm from a lower surface of the first portion 88. In another embodiment, the insertion length L is approximately 11-17 mm. In yet another embodiment, the insertion length L is approximately 11 mm. In yet another embodiment, the insertion length L is approximately 17 mm.

The ventilation shaft 92 is partially defined by an interior wall or dividing wall 100 disposed at the airflow angle relative to the first portion 88. The interior wall 100 is a generally planar wall that partially bounds the ventilation port tube 72 on a first side and partially bounds the tool axis port tube 80 on a second side. Furthermore, the interior wall 100 divides the ventilation port tube 72 from each of the auxiliary port tube 64 and the main insertion tube 60 such that the ventilation port 44 is fluidly coupled to the main shaft 24 by a dedicated airflow tube (i.e., the ventilation port tube 72). In use, the interior wall 100 directs airflow into the main shaft 24 via the ventilation shaft 92. As illustrated in FIG. 4, the main shaft 24 includes a cylindrical outer wall 104 extending through the proximal end 28 to the main insertion port 56. The auxiliary port 48 is coupled to the main shaft 24 by an aperture 108 in the cylindrical outer wall 104. As illustrated, the aperture is a notch 108 within the cylindrical outer wall 104.

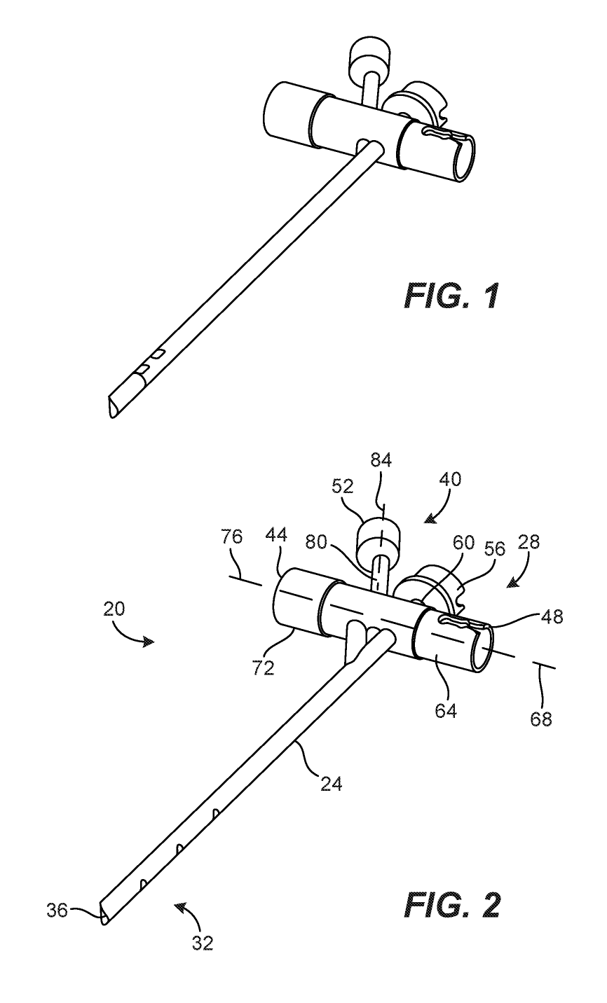

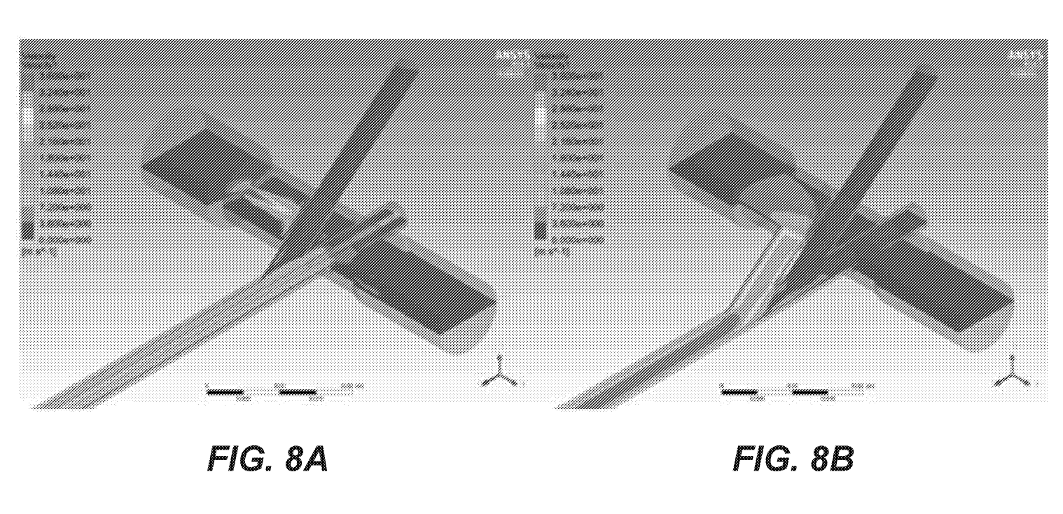

FIG. 8A illustrates an airflow simulation of the prior art bronchoscope illustrated in FIG. 1, and FIG. 8B illustrates an airflow simulation of the bronchoscope illustrated in FIGS. 2-7. As seen in FIG. 8A, air flow velocity enters along the ventilation tube axis at a high velocity, but is significantly decelerated before entering the main shaft 24. In the main shaft 24, air flow velocity is low causing oxygen delivery to a patient to be limited. As illustrated in FIG. 8B, the air flow velocity in the main shaft 24 via the ventilation tube, and more specifically via the ventilation shaft, is significantly higher. The higher velocity of air flow relative to the prior art design leads to an increase in the amount of oxygen delivered to the patient. Testing shows a 93% increase in oxygen delivery to the patient, representing an unexpectedly large increase in oxygen delivery compared to the prior art design. In addition, the increase in oxygen delivery solves a long-felt problem of the prior art design in which inefficient ventilation requires a pause in a procedure to remove the bronchoscope until oxygen levels return to normal. As noted previously, the prior art design increases the time and cost of the operation, and increases the risk of injury to a patient.

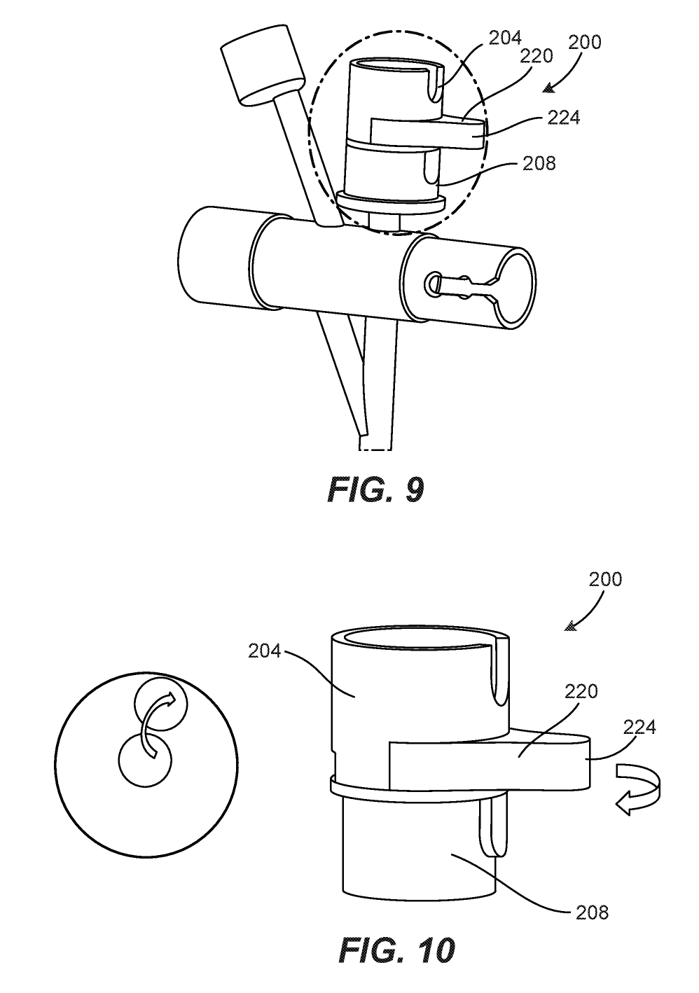

FIGS. 9-12 illustrate an adapter 200 that is coupled to a main insertion port of, for example, the bronchoscope of FIG. 1, the bronchoscope 20 of FIGS. 2-7, or any other bronchoscope. The adapter 200 includes a female end 204 that is coupled to a male end 208. In the illustrated embodiment, the female end 204 is coupled to the male end 208 by a sealing member 212 (e.g., an elastomeric member, an O-ring, etc.) and a fastener 216 (e.g., a screw, a bolt, etc.). However, in other embodiments, the female end 204 and the male end 208 are sealingly coupled via press fitting, a bearing, or other coupling mechanisms. The adapter 200 comprises ABS plastic, but may be constructed from other materials such as various plastics, stainless steel, or other medical grade materials. The adapter 200 may be disposable or reusable. If the adapter 200 is reusable, it is constructed from a material that is able to be sterilized according to standard procedures (e.g., autoclaving, etc.)

With reference to FIG. 10, the female end 204 of the adapter 200 includes a rotary handle 220 defined by a lip 224. The rotary handle 220 is operable by a user to rotate the female end 204 of the adapter 16 relative to the male end 208 of the adapter 200. The rotation of the female end 204 relative to the male end 208 causes a tool that is inserted through the main insertion port to pivot away from a center on the main shaft 24 to a position adjacent a wall of the main shaft 24 (e.g., clockwise with respect to FIG. 10).

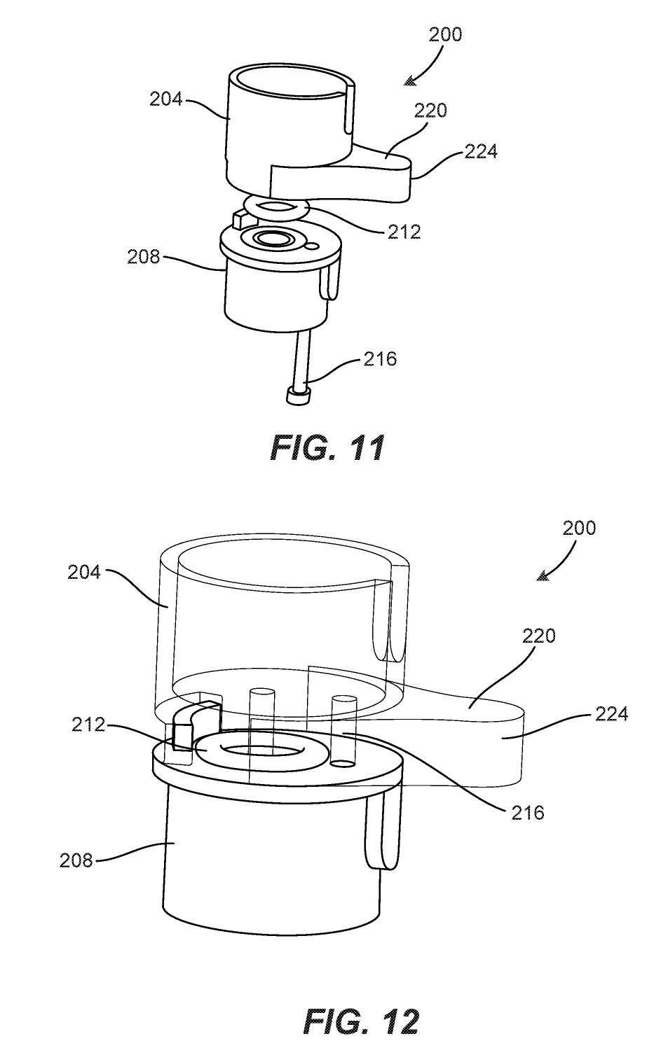

With reference to FIGS. 11-12, the female end 204 is designed to mimic the main insertion port, allowing for it to connect with accessory tools in the same way as other main insertion ports. The male end 208 is coupled to the main insertion port to create a sealed connection. The sealing member 212 prevents leakage between the male end 208 and female end 204, for example, during rotation of the female end 204 relative to the male end 208. In the illustrated embodiment, the pivot point for rotation of the female end 204 relative to the male end 208 is defined by the fastener 216.

Embodiments of the present invention improve upon bronchoscopes used for visualization of the respiratory tract. More specifically, embodiments of the present invention increase delivery of oxygen to patients undergoing treatment.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The various components of the present invention may be constructed generally out of any materials known to be suitable in the art.

Many modifications and other embodiments of the invention set forth herein will come to mind to one skilled in the art to which this invention pertains and having the benefit of the teaching presented in the foregoing descriptions and the associated drawings. Therefore, it should be understood that the inventions are not to be limited to the specific embodiments disclosed and that modifications and other embodiments are intended to be included within the scope of the appended claims. Although specific terms are employed herein, they are used in a generic and descriptive sense only, and not for purposes of limitation.

Various features of the invention are set forth in the following claims.

What is claimed is:

1. A bronchoscope comprising:
a main shaft defined by a hollow tube extending from a proximal end to an open distal end;
a first port at the proximal end and in fluid communication with the main shaft via a first tube, the first port configured to receive one or more operative tools;
a second port at the proximal end and in fluid communication with the main shaft, the second port defining a ventilation port to deliver air to a patient via the main shaft;
a ventilation tube fluidly coupling the second port to the main shaft, wherein the ventilation shaft is fluidly separated from the first tube by a dividing wall.

2. The bronchoscope of claim 1, wherein the ventilation tube is disposed at an oblique angle relative to the main shaft.

3. The bronchoscope of claim 1, wherein the ventilation tube includes a first portion disposed at a first angle relative to the main shaft and a second portion disposed at a second angle relative to the first portion.

4. The bronchoscope of claim 3, wherein the second angle is approximately 60-70 degrees.

5. The bronchoscope of claim 3, wherein the second angle is approximately 63-65 degrees.

6. The bronchoscope of claim 1, wherein the dividing wall extends at an angle relative to the main shaft.

7. The bronchoscope of claim 6, wherein the angle of the dividing wall is 20-30 degrees relative to the main shaft.

8. The bronchoscope of claim 6, wherein the angle of the dividing wall is 25-27 degrees relative to the main shaft.

9. The bronchoscope of claim 1, further including a third port defined at the proximal end that is in fluid communication with the main shaft.

10. The bronchoscope of claim 9, further including a fourth port defined at the proximal end that is in fluid communication with the main shaft, wherein the fourth port is disposed side of the main shaft that is opposite the second port.

11. An adapter for a tool port of a bronchoscope that is continuous with a main shaft of the bronchoscope, comprising:
a first member configured to be coupled to the tool port of the bronchoscope; and
a second member rotationally coupled to the first member, the second member including a port to receive a tool;
wherein rotation of the second member relative to the first member moves the port from a first position to a second position.

12. The adapter of claim 11, wherein the port is aligned with a center of the main shaft in the first position, and is aligned along an axis adjacent a sidewall of the main shaft in the second position.

13. The adapter of claim 11, wherein the adapter includes a fastener coupling the first member to the second member, and the second member rotates relative to the first member along an axis of the fastener.

14. The adapter of claim 11, wherein a seal is disposed between the first member and the second member.

15. The adapter of claim 14, wherein the seal forms a sealing surface about a periphery of the port.

16. A bronchoscope comprising:
a main shaft defined by a hollow tube extending from a proximal end of the bronchoscope to an open distal end of the bronchoscope;
a first port defined at the proximal end that is in fluid communication with the main shaft via a first tube, the first port configured to receive one or more operative tools;
an adapter coupled to the first port, the adapter including
a first member configured to be coupled to the first port, and
a second member rotationally coupled to the first member, the second member including a tool port to receive a tool,
wherein rotation of the second member relative to the first member moves the tool port from a first position to a second position;
a second port defined at the proximal end that is in fluid communication with the main shaft, the second port defining a ventilation port to deliver air to a patient via the main shaft;
a ventilation tube fluidly coupling the second port to the main shaft, wherein the ventilation shaft is fluidly separated from the first tube by a dividing wall.

17. The bronchoscope of claim 16, wherein the ventilation tube includes a first portion disposed at a first angle relative to the main shaft and a second portion disposed at a second angle relative to the first portion, and the second angle is 60-70 degrees.

18. The bronchoscope of claim 17, wherein the second angle is 63-65 degrees.

19. The bronchoscope of claim 16, wherein the tool port is aligned with a center of the main shaft in the first position, and is aligned along an axis adjacent a sidewall of the main shaft in the second position.

20. The bronchoscope of claim 16, the dividing wall extends at an angle of 20-30 degrees relative to the main shaft.
